(19) United States
(12) Patent Application Publication
(10) Pub. No.: US 2023/0034905 A1
(43) Pub. Date: Feb. 2, 2023

(54) ORALLY DISSOLVING FORMULATIONS OF PRUCALOPRIDE

(71) Applicant: SHILPA MEDICARE LIMITED, RAICHUR (IN)

(72) Inventor: RITESH VINOD BIRLA, Hyderabad (IN)

(73) Assignee: SHILPA MEDICARE LTD, RAICHUR (IN)

(21) Appl. No.: 17/782,996

(22) PCT Filed: Dec. 23, 2020
(86) PCT No.: PCT/IB2020/062370

§ 371 Date: Jun. 7, 2022

(30) Foreign Application Priority Data
Dec. 23, 2019 (IN) 201941053389

(51) Int. Cl. A61K 31/4525; A61K 9/00; A61K 47/10; A61K 47/38; A61K 47/26; A61K 47/40

(52) U.S. Cl. CPC A61K 31/4525; A61K 9/006; A61K 47/10; A61K 47/38; A61K 47/26; A61K 47/40

(57) ABSTRACT

The present invention relates to stable orally dissolving formulations e.g., tablets (ODTs) and films (ODFs) comprising prucalopride succinate or pharmaceutically acceptable salts thereof and the process for preparation thereof for the treatment of chronic idiopathic constipation.

### FIELD OF THE INVENTION

The present invention relates to stable orally dissolving formulations e.g., tablets (ODTs) and films (ODFs) comprising prucalopride succinate or pharmaceutically acceptable salts thereof and the process for preparation thereof. The present invention also relates to the method of treating chronic idiopathic constipation by administering orally dissolving formulations comprising prucalopride or pharmaceutically acceptable salts thereof.

### BACKGROUND OF THE INVENTION

Prucalopride, a dihydrobenzofurancarboxamide is a serotonin type 4 (5-HT₄) receptor agonist indicated for the treatment of chronic idiopathic constipation. Prucalopride succinate is structurally represented as

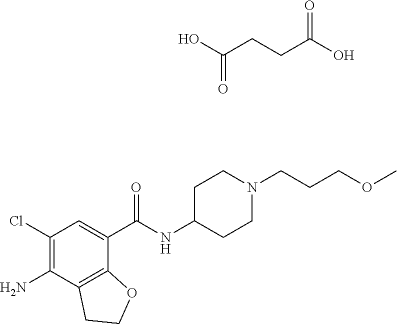 [SMILES: O=C(O)CCC(=O)O ; COCCCN1CCC(NC(=O)c2cc(Cl)c(N)c3c2OCC3)CC1]

Prucalopride is generically described in EP0445862A1, published on Sep. 11, 1991 and is specifically disclosed in WO 96/16060, published on May 90, 1996. Prucalopride succinate film-coated tablets 1 mg and 2 mg are marketed in USA with the brand name Motegrity® by Shire US Inc and with the brand name Resolor® in Europe which comprise the following inactive ingredients as colloidal silicon dioxide, lactose monohydrate, magnesium stearate, and microcrystalline cellulose. The coating for the 1 mg tablet contains hypromellose, lactose monohydrate, polyethylene glycol 3000, titanium dioxide, and triacetin and the coating for the 2 mg tablet contains hypromellose, lactose monohydrate, polyethylene glycol 3000, titanium dioxide, triacetin, red iron oxide, yellow iron oxide, and FD&C Blue #2. Prucalopride succinate has the bitter taste and the two coating formulations are selected to provide taste-masking and colour differentiation between the tablet strengths.

Administration of an oral dosage form is the preferred route of administration for many pharmaceuticals because it provides for easy, low-cost administration. However, some patients such as children or elderly people can have problems when requested to swallow a solid formulation such as a tablet or a capsule.

The inventors of U.S. Pat. No. 6,413,988 have prepared the oral solution comprising prucalopride and preservatives selected from methyl paraben and propyl paraben in accordance with example 22, of EP0445862A2 and administered to test group of 24 human volunteers in a blind study, and found that such oral solution had undesirable organoleptic properties in most volunteers experiencing an anaesthetizing feeling on the tongue. In order to overcome the disadvantage of organoleptic properties the inventors of U.S. Pat. No. 6,413,988 have developed the prucalopride oral solution containing benzoic acid as preservative that do not give anaesthetizing feeling on the tongue, and thus have acceptable organoleptic properties. The prucalopride oral solution as prepared above have the disadvantages of containing preservatives and may cause dosing errors while administration to the patient.

In order to overcome the above disadvantages, there exists a need to develop the stable orally dissolving formulations e.g., tablets (ODTs) and films (ODFs) to improves patient's compliance like organoleptic properties without the use of preservatives and ease of administration without dosing errors.

### SUMMARY OF THE INVENTION

The present invention relates to the stable orally dissolving formulation e.g., tablets (ODTs) and films (ODFs) comprising prucalopride or pharmaceutically acceptable salts thereof.

According to some embodiments of the invention, the present invention provides stable orally dissolving formulation comprising prucalopride or pharmaceutically acceptable salts thereof and a cyclodextrin.

According to other embodiments of the invention, the present invention provides method of treating chronic idiopathic constipation by administering orally dissolving formulations e.g., tablets (ODTs) and films (ODFs) comprising prucalopride or pharmaceutically acceptable salts thereof.

### DETAILED DESCRIPTION OF THE INVENTION

The present invention relates to the stable orally dissolving formulation e.g., tablets (ODTs) and films (ODFs) comprising prucalopride or pharmaceutically acceptable salts thereof and methods for treatment of chronic idiopathic constipation by administering orally dissolving formulations e.g., tablets (ODTs) and films (ODFs) comprising prucalopride or pharmaceutically acceptable salts thereof.

According to some embodiments, the present invention provides stable orally dissolving formulations comprising prucalopride or pharmaceutically acceptable salts thereof and a cyclodextrin.

The term prucalopride as used herein comprises the free base form and the pharmaceutically acceptable acid addition salts thereof. Appropriate acids comprise, for example inorganic acids such as hydrohalic acids, e.g. hydrochloric or hydrobromic acid, sulphuric, nitric, phosphoric and the like acids; or organic acids such as, for example, acetic, propanoic, hyroxyacetic, lactic, pyruvic, oxalic, malonic, succinic, maleic, fumaric, malic, tartaric, citric, methanesulfonic, ethane sulfonic, benzenesulfonic, p-toluene sulfonic, cyclamic, salicylic, p-aminosalicylic, pamoic and the like acids. The term addition salt as used hereinabove also comprises the solvates which prucalopride as well as the salts thereof, are able to form. Such solvates are for examples hydrates, alcoholates and the like. Preferred pharmaceutically acceptable acid addition salt is (1:1) succinic acid addition salt of 4-amino-5-chloro-N-[1-(3-methoxypropyl)piperidin-4-yl]-2,3-dihydrobenzofuran-7-carboxamide (prucalopride succinate).

In some embodiments of the invention, the orally dissolving formulations of the present invention comprises about 0.5% w/w to about 20% w/w of prucalopride or pharmaceutically acceptable salts thereof. In preferred embodiments, the orally dissolving formulations of the present invention comprises about 1% w/w to about 15% w/w of prucalopride succinate and more preferably of about 2% w/w to about 10% w/w based on total weight of orally dissolving formulations.

In first aspect of the invention the present invention provides an orally dissolving film comprising prucalopride or pharmaceutically acceptable salt thereof, a water soluble polymer, a plasticizer, a diluent and a disintegrant wherein prucalopride or pharmaceutically acceptable salt thereof is present in an amount of about 2% w/w to about 10% w/w with respect to the total weight of the film.

As used herein the “water soluble polymer” and variants thereof refer to a polymer that is at least partially soluble in water, and desirably fully or predominantly soluble in water, or absorbs water. Polymers that absorb water are often also referred to as being water swellable polymers, and this term is synonymous for the purposes of the present invention. The materials useful with the present invention may be water soluble at room temperature and other temperatures, such as temperatures exceeding room temperature. Moreover, the materials may be water soluble at pressures less than atmospheric pressure. Desirably, the water soluble polymers have at least 20 percent by weight water uptake. Water soluble polymers having a 25 or greater percent by weight water uptake are also useful. Films or dosage forms of the present invention formed from such water soluble polymers are desirably sufficiently water soluble to be disintegrable/dissolvable upon contact with bodily fluids.

Examples of water soluble polymers include, but are not limited to water-soluble polysaccharides, cellulose polymers or cellulosic derivative polymers, and water soluble synthetic polymers.

In one embodiment, an orally dissolving film formulation of the present invention comprises of about 10% w/w to about 70% w/w, preferably about 15% w/w to about 60% w/w, and more preferably about 20% w/w to about 50% w/w and most preferably of about 25% w/w to about 45% w/w with respect to the total weight of the film.

Water soluble polysaccharides include, but are not limited to alginates such as sodium alginate, carrageenans, guar gum, acacia gum, agar, xanthan gum, gellan gum, arabic gum and related gums (gum ghatti, gum karaya, gum tragancanth), and pectin.

Examples of cellulosic polymers and cellulosic derivative polymers include, but are not limited to alkyl celluloses, hydroxyalkyl celluloses and hydroxyalkyl alkylcelluloses, such as methyl cellulose, hydroxy methylcellulose, hydroxyethyl cellulose, hydroxypropyl cellulose, hydroxyethyl methyl cellulose, hydroxypropyl methylcellulose, hydroxybutyl methylcellulose, cellulose esters and hydroxyalkylcellulose esters such as cellulose acetate phthalate; carboxy alkylcelluloses, carboxyalkyl alkylcelluloses, carboxyalkyl cellulose esters such as carboxymethyl cellulose and their alkali metal salts. In some preferred embodiments, the cellulose polymer and cellulosic derivative polymers include, but are not limited to, methyl cellulose, ethyl cellulose, hydroxypropyl ethylcellulose, hydroxyethyl cellulose, hydroxypropyl methylcellulose, cellulose acetate phthalate, hydroxypropyl methyl cellulose phthalate, and combinations thereof. The more preferred cellulose polymer is selected from the group consisting of hydroxypropyl methylcellulose, hydroxypropyl cellulose and carboxymethyl cellulose. The most preferred cellulose polymer is hydroxypropyl methylcellulose.

In preferred embodiments, the orally dissolving film dosage form includes one or more cellulose polymers (water soluble polymer) selected from the group consisting of hydroxypropyl methylcellulose, hydroxypropyl cellulose and carboxymethyl cellulose.

In preferred embodiments, the total amount of the hydroxypropyl methylcellulose present in the film dosage form ranges from about 10% w/w to about 70% w/w, preferably about 15% w/w to about 60% w/w, more preferably about 20% w/w to about 50% w/w and most preferably of about 25% w/w to about 45% w/w with respect to the total weight of the film.

Synthetic polymers include, but are not limited to polyacrylic acids and polyacrylic acid esters, polymethacrylic acids and polymethacrylic acid esters, polyalkylene oxides, such as polyethylene oxide, polyvinylacetates, polyvinylalcohols, polyvinylacetatephthalates (PVAP), polyvinylpyrrolidone (PVP), polyvinyl acetate copolymers, and polycrotonic acids; also suitable are phthalated gelatin, gelatin succinate, crosslinked gelatin, shellac, water soluble chemical derivatives of starch, cationically modified acrylates and methacrylates possessing, for example, a tertiary or quaternary amino group, such as the diethylaminoethyl group, which may be quarternized if desired.

In embodiments of invention plasticizers used in the present invention include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sodium diethylsulfosuccinate, triethyl citrate, tributyl citrate, and the like. More preferably the plasctizer used in the present invention is selected from the group consisting of glycerol, propylene glycol and triethyl citrate. Glycerol is most preferred plasctizer and is added in concentrations ranging from about 0.5% w/w to about 30% w/w, more preferably from about 5% w/w to about 25% w/w and most preferably from about 10% w/w to about 20% w/w with respect to the total weight of the film.

In embodiments of the present invention diluents used for the preparation of orally dissolving film dosage form of present invention are selected from mannitol, microcrystalline cellulose (for example, microcrystalline cellulose available under the trade mark Avicel®), starches or modified starches (including potato starch, corn starch, maize starch and rice starch) and tribasic calcium phosphate. In preferred embodiment diluents used in the present invention are selected from the group consisting of mannitol and microcrystalline cellulose. Diluent used in the present invention ranges from about 1% w/w to about 30% w/w, preferably about 2% w/w to about 10% w/w with respect to the total weight of the film. Most preferably, diluent used in the present invention is microcrystalline cellulose in an amount of about 2% w/w to about 10% w/w with respect to the total weight of the film.

In embodiments of the present invention disintegrants used for the preparation of orally dissolving film dosage form of the present invention are selected from group consisting of silicified microcrystalline cellulose, croscarmellose sodium and sodium starch glycolate. The most preferably used disintegrant in the present invention is silicified microcrystalline cellulose. The silicified microcrystalline cellulose is an intimate mixture of colloidal silicon dioxide with microcrystalline cellulose as described in U.S. Pat. No. 5,585,115. The amount of the silicified microcrystalline cellulose used in the present invention for preparation of the film is in an amount of about 5% w/w to about 20% w/w and most preferably in an amount of about 10% w/w to about 20% w/w with respect to the total weight of the film.

The present inventors have surprisingly discovered that the use of about 5% w/w to about 20% w/w, more preferably about 10% w/w to about 20% w/w of silicified microcrystalline cellulose has enhanced the dissolution of the film dosage form compared to film dosage form without the silicified microcrystalline or less than 5% w/w or more than 20% w/w of silicified microcrystalline cellulose.

In a further embodiment, the present invention relates to an orally dissolving film comprising
(a) about 2% w/w to about 10% w/w of prucalopride succinate;
(b) about 25% w/w to about 45% w/w of water soluble polymer;
(c) about 10% w/w to about 20% w/w of plasticizer;
(d) about 2% w/w to about 10% w/w of diluent; and
(e) about 10% w/w to about 20% w/w of disintegrant.

In a still further embodiment, the present invention relates to an orally dissolving film comprising sweeteners selected from glucose (corn syrup), dextrose, invert sugar, fructose, and combinations thereof; saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, xylitol, and erythritol. Also contemplated are hydrogenated starch hydrolysates and the synthetic sweetener 3,6-dihydro-6-methyl-1-1-1,2,3-o-xathiazin-4-one-2,2-dioxide, particularly the potassium salt (acesulfame-K), ammoniated glycyrrhizin and monoammonium glycyrrhizinate, and sodium and calcium salts thereof, and natural intensive sweeteners. Other sweeteners may also be used. The more preferably used sweeteners used in the present invention are selected from the group consisting of sucralose, aspartame and sorbitol. The most preferably used sweetener in the present invention is sucralose. The amount of sucralose used in the present invention is of about 1% w/w to about 20% w/w, and most preferably in amount of about 2% w/w to about 10% w/w with respect to the total weight of the film.

In still further another embodiment, the present invention relates to an orally dissolving film comprising
(a) about 2% w/w to about 10% w/w of prucalopride succinate;
(b) about 25% w/w to about 45% w/w of hydroxypropyl methylcellulose;
(c) about 10% w/w to about 20% w/w of glycerol;
(d) about 2% w/w to about 10% w/w of microcrystalline cellulose;
(e) about 10% w/w to about 20% w/w of silicified microcrystalline cellulose; and
(f) a sweetener selected from group consisting of sucralose, aspartame and sorbitol.

In another embodiment, the present invention relates to the orally dissolving film comprising the flavouring agents may be chosen from natural and synthetic flavouring liquids. Examples of flavouring agents includes mint oils (peppermint), cocoa, and citrus oils such as lemon, orange, grape, lime and grapefruit and fruit essences including apple, pear, peach, grape, strawberry, raspberry, cherry, plum, pineapple, apricot or other fruit flavours. Other useful flavourings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like. The most preferably used flavouring agents used in the present invention is a mixture of orange flavour and peppermint flavour. Orange flavour is present in an amount of about 2% w/w to about 15% w/w and more preferably in an amount of about 5% w/w to about 12% w/w with respect to the total weight of the film. Peppermint flavour is present in an amount of about 0.5% w/w to about 10% w/w and more preferably in amount of about 1% w/w to about 5% w/w with respect to the total weight of the film.

In still further another embodiment, the present invention relates to an orally dissolving film comprising
(a) about 2% w/w to about 10% w/w of prucalopride succinate;
(b) about 25% w/w to about 45% w/w of hydroxypropyl methylcellulose;
(c) about 10% w/w to about 20% w/w of glycerol;
(d) about 2% w/w to about 10% w/w of microcrystalline cellulose;
(e) about 10% w/w to about 20% w/w of silicified microcrystalline cellulose;
(f) about 2% w/w to about 10% w/w of sucralose; and
(g) an optional flavouring agent mixture consisting of orange flavour and peppermint flavour.

The inventors of the present invention have surprisingly found that about 2% w/w to about 10% w/w of sucralose and a flavouring agent mixture of orange flavour and peppermint flavour has effectively masked the bitter taste of the prucalopride succinate active ingredient.

In a still further embodiment, the present invention relates to an orally dissolving film comprising
(a) about 2% w/w to about 10% w/w of prucalopride succinate;
(b) about 25% w/w to about 45% w/w of hydroxypropyl methylcellulose;
(c) about 10% w/w to about 20% w/w of glycerol;
(d) about 2% w/w to about 10% w/w of microcrystalline cellulose;
(e) about 10% w/w to about 20% w/w of silicified microcrystalline cellulose;
(f) about 2% w/w to about 10% w/w of sucralose;
(g) about 5% w/w to about 12% w/w of orange flavour and
(h) about 1% w/w to about 5% w/w of peppermint flavour.

In the further embodiment, the film dosage forms of the present invention comprise colorants (colouring agents) which include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide. Other examples of coloring agents include known azo dyes, organic or inorganic pigments (Yellow iron oxide), or coloring agents of natural origin.

In a still specific embodiment the present invention relates to an orally dissolving film consisting of
(a) about 2% w/w to about 10% w/w of prucalopride succinate;
(b) about 25% w/w to about 45% w/w of hydroxypropyl methylcellulose;
(c) about 10% w/w to about 20% w/w of glycerol;
(d) about 2% w/w to about 10% w/w of microcrystalline cellulose;
(e) about 10% w/w to about 20% w/w of silicified microcrystalline cellulose;
(f) about 2% w/w to about 10% w/w of sucralose;
(g) about 5% w/w to about 12% w/w of orange flavour;
(h) about 1% w/w to about 5% w/w of peppermint flavour and
(i) a coloring agent.

In embodiments of the invention, the orally dissolving film of the present invention is free of preservatives and antioxidants.

In embodiments of the invention, the present invention provides an orally dissolving film comprising prucalopride or pharmaceutically acceptable salt thereof, a water soluble polymer, a plasticizer, a diluent and a disintegrant wherein prucalopride or pharmaceutically acceptable salt thereof is present in an amount of about 2% w/w to about 10% w/w with respect to the total weight of the film, wherein the film is free of preservatives and antioxidants.

Ina further embodiment, the present invention relates to an orally dissolving film comprising
(a) about 2% w/w to about 10% w/w of prucalopride succinate;
(b) about 25% w/w to about 45% w/w of hydroxypropyl methylcellulose;
(c) about 10% w/w to about 20% w/w of glycerol;
(d) about 2% w/w to about 10% w/w of microcrystalline cellulose; and
(e) about 10% w/w to about 20% w/w of silicified microcrystalline cellulose; wherein the film is free of preservatives and antioxidants.

In a still further specific embodiment the present invention relates to an orally dissolving film consisting of
(a) about 2% w/w to about 10% w/w of prucalopride succinate;
(b) about 25% w/w to about 45% w/w of hydroxypropyl methylcellulose;
(c) about 10% w/w to about 20% w/w of glycerol;
(d) about 2% w/w to about 10% w/w of microcrystalline cellulose;
(e) about 10% w/w to about 20% w/w of silicified microcrystalline cellulose;
(f) about 2% w/w to about 10% w/w of sucralose;
(g) about 5% w/w to about 12% w/w of orange flavour;
(h) about 1% w/w to about 5% w/w of peppermint flavour and
(i) a coloring agent;
   wherein the film is free of preservatives and antioxidants.

In another embodiment of the present invention, the film dispersion time of the prucalopride film of the present invention is rapid within ten seconds.

In one of the embodiments of the present invention, the dissolution time of the prucalopride succinate film of the present invention is comparable to Resolor® tablets.

In another embodiment of the present invention it provides a process for preparing a pharmaceutical film composition of prucalopride succinate comprising the steps of:
(a) providing a solution comprising prucalopride succinate, sweeteners and diluents dissolved in purified water;
(b) preparing separately an aqueous dispersion of water-soluble polymer;
(c) transferring the dispersion of step (b) into step (a) solution;
(d) adding plasticizers into step (c) solution, followed by dispersing colorants and flavouring agents; and
(e) casting the film composition onto a polyester film.

In another aspect the present invention relates to a pharmaceutical composition comprising prucalopride or pharmaceutically acceptable salt thereof, a cyclodextrin, a water soluble polymer, a plasticizer, a diluent and a disintegrant wherein prucalopride or pharmaceutically acceptable salt thereof is present in an amount of about 2% w/w to about 10% w/w with respect to the total weight of the film.

The cyclodextrin are selected from α, β, γ cyclodextrin and derivatives thereof. Cyclodextrins for use in the present invention include the natural cyclodextrins and their derivatives, including the alkylated, hydroxyalkylated, sulfobutyl derivatives and the branched cyclodextrin derivatives bearing sugar residues of special interest. Especially useful herein are the hydroxyethyl, hydroxypropyl (including 2-hydroxypropyl and 3-hydroxypropyl), sulfobutyl, and dihydroxypropyl ethers, their corresponding mixed ethers and further mixed ethers with methyl or ethyl groups, such as methyl-hydroxyethyl, ethyl-hydroxyethyl and ethyl-hydroxypropyl ethers of α, β, γ cyclodextrin. Specific cyclodextrin derivatives for use herein are selected from group consisting of methyl-α-cyclodextrin, hydroxyethyl-α-cyclodextrin, hydroxypropyl-α-cyclodextrin, dihydroxypropyl-α-cyclodextrin, methyl-β-cyclodextrin, hydroxyethyl-β-cyclodextrin, hydroxypropyl-β-cyclodextrin, dihydroxypropyl-β-cyclodextrin, sulfobutylether-β-cyclodextrin, methyl-γ-cyclodextrin, hydroxyethyl-γ-cyclodextrin, hydroxypropyl-γ-cyclodextrin, and dihydroxypropyl-γ-cyclodextrin. Most preferably the cyclodextrin is selected from the group consisting of hydroxypropyl-β-cyclodextrin and sulfobutylether-β-cyclodextrin.

The inventors of the present invention have surprisingly found that the orally dissolving formulations of present invention comprising prucalopride succinate and cyclodextrin derivatives have the total impurities less than about 2.0%, more preferably less than about 1.0%, when stored at 40° C./75% RH (accelerated storage conditions) for at least 3 months.

In a still further embodiment, the present invention provides the orally dissolving formulations comprising prucalopride succinate and cyclodextrin derivatives, wherein total amount of impurities present in the formulation is less than about 2.0%.

In more preferred embodiment, the present invention provides the orally dissolving formulations comprising prucalopride succinate and (2-hydroxypropyl-β-cyclodextrin, wherein total amount of impurities present in the formulation is less than about 2.0%.

In some embodiments of the invention, the orally dissolving formulations of the present invention comprises about 10% w/w to about 60% w/w of cyclodextrin derivatives. In preferred embodiments, the orally dissolving formulations of the present invention comprises about 20% w/w to about 55% w/w of cyclodextrin derivatives and more preferably about 25% w/w to about 50% w/w of cyclodextrin derivatives based on the total weight of the dosage form.

In some embodiments of the invention, the orally dissolving formulations of the present invention comprises about 10% w/w to about 60% w/w of 2-hydroxypropyl-β-cyclodextrin. In preferred embodiments, the orally dissolving formulations of the present invention comprises about 20% w/w to about 55% w/w of 2-hydroxypropyl-β-cyclodextrin and more preferably about 25% w/w to about 50% w/w of 2-hydroxypropyl-β-cyclodextrin based on the total weight of the dosage form.

In a further embodiment, the present invention relates to an orally dissolving film comprising
(a) about 2% w/w to about 10% w/w of prucalopride succinate;
(b) about 25% w/w to about 50% w/w hydroxypropyl-f-cyclodextrin;
(c) about 25% w/w to about 45% w/w of hydroxypropyl methylcellulose;
(d) about 5% w/w to about 20% w/w of glycerol;
(e) about 2% w/w to about 10% w/w of microcrystalline cellulose; and
(f) about 5% w/w to about 20% w/w of silicified microcrystalline cellulose.

Another embodiment of the present invention it provides a process for preparing a pharmaceutical film composition of prucalopride succinate comprising the steps of
(a) providing a solution of cyclodextrin derivatives in purified water;
(b) adding prucalopride succinate, sweeteners and diluents into step (a) solution;
(c) preparing separately an aqueous dispersion of water-soluble polymer;
(d) transferring the dispersion of step (c) into step (b) solution;
(e) adding plasticizers into step (d) solution, followed by dispersing colorants and flavouring agent; and
(f) obtaining the film composition onto a polyester film.

Another embodiment of the present invention it provides a process for preparing a pharmaceutical film composition of prucalopride succinate comprising the steps of
(a) providing a solution of hydroxypropyl-β-cyclodextrin in purified water;
(b) adding prucalopride succinate, sucralose, microcrystalline cellulose and silicified microcrystalline cellulose into step (a) solution;
(c) preparing separately an aqueous dispersion of hydroxypropylmethyl cellulose polymer;
(d) transferring the dispersion of step (c) into step (b) solution; and
(e) adding plasticizers into step (d) solution, followed by dispersing colorants and flavorants; and
(f) obtaining the film composition onto a polyester film.

The example given below serve to illustrate embodiments of the present invention. However, they do not intend to limit the scope of present invention.

### Example 1: Orally Dissolving Film Composition of Prucalopride Succinate

| Ingredients | mg/strip | % w/w |
|---|---|---|
| Prucalopride Succinate | 2.642 | 7.0% |
| Sucralose | 2 | 5.3% |
| Microcrystalline cellulose | 2 | 5.3% |
| Silicified microcrystalline cellulose | 5 | 13.2% |
| Orange Flavor | 4 | 10.5% |
| Peppermint Flavor | 1 | 2.6% |
| Hydroxypropyl methylcellulose | 15.05 | 39.6% |
| Iron oxide of yellow | 0.3 | 0.8% |
| Glycerol | 6 | 15.8% |
| Purified water | q. s | q.s |
| Strip (film) weight | 38.00 | |

### Process for Preparation:

a) Prucalopride succinate, sucralose, microcrystalline cellulose, silicified microcrystalline cellulose was dissolved in purified water and stirred to prepare a solution of prucalopride succinate.
b) Aqueous dispersion of hydroxypropylmethyl cellulose polymer was prepared by dispersing hydroxypropylmethyl cellulose in purified water at 37° C.±5° C. temperature and stirred.
c) Aqueous dispersion of hydroxypropylmethyl cellulose was dispersed in prucalopride solution.
d) Glycerol, orange flavor, peppermint flavor and iron oxide yellow was added to contents of step c and
e) the contents of step d were layered onto polyester film, to form the film, dried, slitted and packed.

Dissolution profile comparison: The dissolution profile comparison of Resolor® Tablets 2 mg against example-1 prucalopride film of the present invention (media pH 1.2 0.1 N HCl; pH 4.5 buffer and pH 6.8 buffer in USP Apparatus 1 stirred at 100 rpm) is represented in the following Table-1.

TABLE 1

| Dissolution | Sampling | Resolor ® | Example-1 |
|---|---|---|---|
| Media | Times | Tablets | Film |
| pH 1.2 | 5 min | 64 | 54 |
| (0.1 N HCL with | 10 min | 93 | 77 |
| 900 ml, Apparatus | 15 min | 96 | 90 |
| 1, 100 rpm) | 20 min | 96 | 96 |
| | 30 min | 96 | 98 |
| | 45 min | 96 | 99 |
| pH 4.5 Buffer | 5 min | 71 | 98 |
| (4.5 buffer with | 10 min | 94 | 99 |
| 900 ml, Apparatus | 15 min | 94 | 99 |
| 1, 100 rpm) | 20 min | 94 | 99 |
| | 30 min | 94 | 99 |
| | 45 min | 94 | 101 |
| pH 6.8 Buffer | 5 min | 71 | 96 |
| (6.8 buffer with | 10 min | 94 | 96 |
| 900 ml, Apparatus | 15 min | 96 | 99 |
| 1, 100 rpm) | 20 min | 96 | 99 |
| | 30 min | 96 | 100 |
| | 45 min | 96 | 100 |

### Example 2 and 3: Orally Dissolving Film Compositions of Prucalopride Succinate

| | Example-2 | | Example-3 | |
|---|---|---|---|---|
| Ingredients | mg/strip | % w/w | mg/strip | % w/w |
| Prucalopride Succinate | 2.642 | 7.8% | 2.642 | 6.1% |
| Sucralose | 2 | 5.9% | 2 | 4.7% |
| Microcrystalline cellulose | 2 | 5.9% | 2 | 4.7% |
| Silicified microcrystalline | 1 | 2.9% | 10 | 23.3% |
| cellulose | | | | |
| Orange Flavor | 4 | 11.8% | 4 | 9.3% |
| Peppermint Flavor | 1 | 2.9% | 1 | 2.3% |
| Hydroxypropyl methylcellulose | 15.05 | 44.3% | 15.05 | 35% |
| Iron oxide of yellow | 0.3 | 0.7% | 0.3 | 0.7% |
| Glycerol | 6 | 17.7% | 6 | 14% |
| Purified water | q. s | q.s | q. s | q.s |
| Strip (film) weight | 34.00 | | 43.00 | |

The films of prucalopride of example 2 and 3 are prepared as per the process as disclosed in example-1.

Dissolution profile: The dissolution profile of example 2 and example 3 in pH 1.2 Media 0.1 N HCl is represented in following Table-2.

TABLE 2

| Dissolution | Sampling | Films of | Films of |
|---|---|---|---|
| Media | Times | Example-2 | Example-3 |
| pH 1.2 | 5 min | 35 | 45 |
| (0.1 N HCL with | 10 min | 59 | 56 |
| 900 ml, Apparatus | 15 min | 68 | 72 |
| 1, 100 rpm) | 20 min | 75 | 84 |
| | 30 min | 80 | 92 |
| | 45 min | 86 | 97 |

### Example 4: Dissolution Profiles with Varying Concentrations of Silicified Microcrystalline Cellulose

The dissolution profile with varying concentration of silicified microcrystalline cellulose 0% w/w, 2% w/w, 5% w/w, 8% w/w, 10% w/w, 15% w/w, 20% w/w and 25% w/w of the film in in pH 1.2 Media 0.1 N HCl is represented in following Table-3.

TABLE 3

| Varying concentration of silicified | 5 | 10 | 15 | 20 | 30 |
|---|---|---|---|---|---|
| microcrystalline cellulose | min | min | min | min | min |
| Silicified microcrystalline cellulose not | 15 | 35 | 59 | 68 | 75 |
| present in the film (0% w/w) | | | | | |
| Silicified microcrystalline cellulose | 32 | 55 | 62 | 72 | 79 |
| (2% w/w) | | | | | |
| Silicified microcrystalline cellulose | 49 | 72 | 85 | 92 | 95 |
| (5% w/w) | | | | | |
| Silicified microcrystalline cellulose | 52 | 75 | 89 | 95 | 97 |
| (10% w/w) | | | | | |
| Silicified microcrystalline cellulose | 54 | 77 | 92 | 98 | 99 |
| (15% w/w) | | | | | |
| Silicified microcrystalline cellulose | 50 | 70 | 87 | 93 | 96 |
| (20% w/w) | | | | | |
| Silicified microcrystalline cellulose | 47 | 55 | 75 | 85 | 94 |
| (25% w/w) | | | | | |

From the above table, the inventors of the present invention have unexpectedly found that prucalopride film composition with the specific concentration range of 5% w/w to about 20% w/w of silicified microcrystalline cellulose, more specifically 10% w/w to about 20% w/w of silicified microcrystalline cellulose based on the total weight of the film composition is found to be critical to have the high dissolution which is equivalent to the dissolution of the Resolor® Tablets, when compared to the films having less than 5% w/w of silicified microcrystalline cellulose and more than 20% w/w of silicified microcrystalline cellulose.

### Example 5: Taste Evaluation of the Prucalopride Films with Varying Concentrations of Different Sweeteners and Various Combinations of Flavoring Agents

Different film compositions containing certain amounts of sucralose in combination with different flavoring agents are evaluated for taste. Ten men and women aged 25 to 40 years were allowed to feel the taste of the different film compositions, the taste was scored based on the following criteria and those scores were averaged (the values were rounded off to two decimals). The taste score is marked for each individual based on following criteria.
0: No bitter taste
1: Tasteless
2: Bitter taste was perceptible
3: Slightly bitter taste was felt
4: Bitter taste was felt
5: Strong bitter taste was felt.

The results of this test are represented in following Table-4.

TABLE 4

| | | Bitter taste |
|---|---|---|
| Sweetener | Flavouring agent | score |
| 2% w/w sorbitol | 10.5% w/w Pine apple flavour | 4.8 |
| 5% w/w sorbitol | 10.5% w/w Pine apple | 4.3 |
| | flavour + 2.6% peppermint | |
| | flavour | |
| 5% w/w sucralose | 10.5% w/w Pine apple | 1.8 |
| | flavour + 2.6% peppermint | |
| | flavour | |
| 1% w/w sucralose | 10.5% orange flavour + | 2.6 |
| | 2.6% peppermint flavour | |
| 3% w/w sucralose | 10.5% orange flavour + | 0.7 |
| | 2.6% peppermint flavour | |
| 5.3% w/w sucralose | 10.5% orange flavour + | 0.2 |
| | 2.6% peppermint flavour | |
| 7% w/w sucralose | 10.5% orange flavour + | 0.8 |
| | 2.6% peppermint flavour | |
| 10% w/w sucralose | 10.5% orange flavour + | 0.95 |
| | 2.6% peppermint flavour | |
| 12% w/w sucralose | 10.5% orange flavour + | 2.2 |
| | 2.6% peppermint flavour | |
| 5% w/w aspartame | 10.5% w/w Pine apple | 3.6 |
| | flavour + 2.6% peppermint | |
| | flavour | |
| 5% w/w aspartame | 10.5% w/w Pine apple | 3.8 |
| | flavour + 2.6% peppermint | |
| | flavour | |
| 5% w/w aspartame | 10.5% orange flavour + | 3.9 |
| | 2.6% peppermint flavour | |

From the above table the inventors of the present invention have surprisingly found that about 2% w/w to about 10% w/w of sucralose and a flavouring agent mixture of orange flavour and peppermint flavour has effectively masked the bitter taste of the prucalopride succinate active ingredient.

### Example 6 and 7: Orally Dissolving Film Composition of Prucalopride Succinate

| | | Example-6 | Example-7 |
|---|---|---|---|
| S.NO. | Ingredients | mg/Strip | mg/Strip |
| 1 | Prucalopride Succinate | 2.642 | 2.642 |
| 2 | 2-hydroxypropyl-β-cyclodextrin, | 40 | 20 |
| 3 | Sucralose | 2 | 2 |
| 4 | Microcrystalline Cellulose | 2 | 2 |
| 5 | Silicified Microcrystalline Cellulose | 5 | 5 |
| 6 | Orange flavor | 4 | 4 |
| 7 | Peppermint flavor | 1 | 1 |
| 8 | Hydroxypropylmethyl cellulose | 25.06 | 25.6 |
| 9 | Iron oxide of yellow | 0.3 | 0.3 |
| 10 | Glycerol | 6.00 | 6 |
| 11 | Purified Water | q. s | q. s |

| | | |
|---|---|---|
| Strip (film) weight | 90.0 | 68.00 |

Process for Preparation:
a) Prucalopride succinate, 2-hydroxypropyl-β-cyclodextrin, sucralose, microcrystalline cellulose, silicified microcrystalline cellulose was dissolved in purified water and stirred to prepare a solution of prucalopride succinate.
b) Aqueous dispersion of hydroxypropylmethyl cellulose polymer was prepared by dispersing hydroxypropylmethyl cellulose in purified water at 37° C.±5° C. temperature and stirred.
c) Aqueous dispersion of hydroxypropylmethyl cellulose was dispersed in prucalopride solution.
d) Glycerol, orange flavor, peppermint flavor and iron oxide yellow was added to contents of step c and
e) the contents of step d were layered onto polyester film, to form the film, dried, slitted and packed.

The film dosage form of prucalopride succinate of example-2 and example-3 was stored at 40° C./75% RH (accelerated temperature) for about 2 months and 3 months respectively; results are tabulated in Table-5 and 6 respectively.

TABLE 5
(Example-6)

| Time/Testing | | 40° C./75% RH | |
|---|---|---|---|
| Parameter | Initial | 1 M | 2 M |
| Assay (%) | 98.9 | 98.8 | 98.5 |
| Impurity-1 | ND | 0.05 | 0.08 |
| Impurity-2 | 0.02 | 0.11 | 0.14 |
| Impurity-3 | 0.02 | 0.03 | 0.04 |
| Impurity-4 | ND | 0.09 | 0.17 |
| Unknown impurity | 0.02 | 0.02 | 0.03 |
| Total impurities (%) | 0.06 | 0.36 | 0.50 |

TABLE 6
(Example-7)

| Time/Testing | | 40° C./75% RH | | |
|---|---|---|---|---|
| Parameter | Initial | 1 M | 2 M | 3 M |
| Assay (%) | 97.7 | 97.5 | 96.2 | 97.7 |
| Impurity-1 | ND | 0.04 | 0.04 | 0.08 |
| lmpurity-2 | 0.04 | 0.08 | 0.14 | 0.19 |
| Impurity-3 | ND | 0.03 | 0.04 | 0.04 |
| Impurity-4 | 0.13 | 0.42 | 0.57 | 0.68 |
| Unknown impurity | ND | 0.11 | 0.12 | 0.15 |
| Total impurities (%) | 0.17 | 0.74 | 0.93 | 1.17 |

We claim:

1. An orally dissolving film comprising prucalopride or pharmaceutically acceptable salt thereof, a water soluble polymer, a plasticizer, a diluent and a disintegrant wherein prucalopride or pharmaceutically acceptable salt thereof is present in an amount of about 2% w/w to about 10% w/w with respect to the total weight of the film.

2. The orally dissolving film as claimed in claim 1, comprising
(a) about 2% w/w to about 10% w/w of prucalopride succinate;
(b) about 25% w/w to about 45% w/w of water soluble polymer;
(c) about 10% w/w to about 20% w/w of plasticizer;
(d) about 2% w/w to about 10% w/w of diluent; and
(e) about 10% w/w to about 20% w/w of disintegrant.

3. The orally dissolving film as claimed in claim 2, wherein water soluble polymer is selected from the group consisting of hydroxypropyl methylcellulose, hydroxypropyl cellulose and carboxymethyl cellulose.

4. The orally dissolving film as claimed in claim 2, wherein plasticizer is selected from the group consisting of glycerol, propylene glycol and triethyl citrate.

5. The orally dissolving film as claimed in claim 2, wherein diluent is selected from the group consisting of mannitol and microcrystalline cellulose.

6. The orally dissolving film as claimed in claim 2, wherein disintegrant is selected from the group consisting of silicified microcrystalline cellulose, croscarmellose sodium and sodium starch glycolate.

7. An orally dissolving film comprising
(a) about 2% w/w to about 10% w/w of prucalopride succinate;
(b) about 25% w/w to about 45% w/w of hydroxypropyl methylcellulose;
(c) about 10% w/w to about 20% w/w of glycerol;
(d) about 2% w/w to about 10% w/w of microcrystalline cellulose;
(e) about 10% w/w to about 20% w/w of silicified microcrystalline cellulose;
(f) a sweetener selected from group consisting of sucralose, aspartame and sorbitol.

8. An orally dissolving film comprising prucalopride or pharmaceutically acceptable salt thereof, a cyclodextrin, a water soluble polymer, a plasticizer, a diluent and a disintegrant wherein prucalopride or pharmaceutically acceptable salt thereof is present in an amount of about 2% w/w to about 10% w/w with respect to the total weight of the film.

9. The orally dissolving film as claimed in claim 8, wherein cyclodextrin is selected from group consisting of hydroxypropyl-β-cyclodextrin and sulfobutylether-β-cyclodextrin.

10. The orally dissolving film as claimed in claim 8, comprising
(a) about 2% w/w to about 10% w/w of prucalopride succinate;
(b) about 25% w/w to about 50% w/w hydroxypropyl-β-cyclodextrin;
(c) about 25% w/w to about 45% w/w of hydroxypropyl methylcellulose;
(d) about 5% w/w to about 20% w/w of glycerol;
(e) about 2% w/w to about 10% w/w of microcrystalline cellulose; and
(f) about 5% w/w to about 20% w/w of silicified microcrystalline cellulose.
